(19) United States
(12) Patent Application Publication
(10) Pub. No.: US 2011/0288128 A1
(43) Pub. Date: Nov. 24, 2011

(54) Oral Transmucosal Administration of Sufentanil

(75) Inventors: Pamela Palmer, San Francisco, CA (US); Shamim Pushpala, Sunnyvale, CA (US); Evangeline Cruz, Hayward, CA (US); Larry Hamel, Mountain View, CA (US); Linda Judge, Belmont, CA (US)

(73) Assignee: AcelRx Pharmaceuticals, Inc., Redwood City, CA (US)

(21) Appl. No.: 12/784,653

(22) Filed: May 21, 2010

(51) Int. Cl. A61K 31/4535; A61P 25/04; C07D 409/06

(52) U.S. Cl. 514/326; 546/213

(57) ABSTRACT

Compositions and methods for administration of sufentanil-containing drug formulations to the oral mucosa of a subject are disclosed.

### FIELD OF THE INVENTION

The invention relates to formulations and methods for oral transmucosal administration of sufentanil, which are effective to result in a novel pharmacokinetic profile, in addition to providing advantages over existing pain treatment modalities with respect to safety and efficacy. The invention further relates to compositions and methods for treatment of acute pain and breakthrough pain using a multi-dose dispenser or a SDA.

### BACKGROUND OF THE INVENTION

Oral dosage forms account for approximately eighty percent of all the drug dosage forms on the market. They are non-invasive, easily administered and have high patient compliance. Orally administered therapeutic agents, however, must be transported to the stomach and small intestine for absorption across the gastrointestinal (GI) mucosal membranes into the blood. The efficiency of absorption of a drug following oral administration can be low because of drug dissolution, metabolism within the GI tract and first-pass metabolism within the liver, resulting in relatively lengthy onset times or erratic absorption characteristics that are not well-suited to control acute disorders or breakthrough pain events. The majority of oral dosage forms on the market are designed for GI delivery.

Oral transmucosal delivery offers a number of advantages in that it can provide a shorter onset time to maximal plasma concentration (Cₘₐₓ) than oral delivery, in particular for lipophilic drugs. This is because the drug rapidly passes directly and efficiently through the epithelium of the highly vascularized mucosal tissue to the plasma, thus rapidly reaching the circulation while avoiding slower, often inefficient and variable GI uptake. It is therefore advantageous for a drug to be delivered through the mucous membranes of the oral cavity.

However, frequently the key risk associated with oral transmucosal delivery is the enhanced potential for swallowing the medication owing to the continuous generation, backward flow and swallowing of the saliva. This becomes a particular risk when the dosage forms employed are large enough to produce a significant saliva response, which, in turn, leads to swallowing of drug and/or loss of adherence of the dosage form to the oral mucosa.

Various solid dosage forms, such as sublingual tablets, troches, lozenges, lozenges-on-a-stick, chewing gums, and buccal patches have been used to deliver drugs via the oral mucosal tissue. Solid dosage forms such as lozenges and tablets have been used for sublingual delivery of drugs such as nitroglycerin.

Reproducible and effective drug delivery technology represents an area of active research, in particular, as it applies to controlled substances such as opioids.

A need exists for improved drug compositions, methods and systems for the treatment of pain, in particular, safe, efficacious, patient-controlled administration. This is particularly relevant to the treatment of acute, intermittent and breakthrough pain.

The present invention addresses this need.

### BRIEF SUMMARY OF THE INVENTION

Pharmaceutical composition comprising sufentanil which have an oral transmucosal bioavailability of from about 50% to about 90% and a gastrointestinal (GI) bioavailability of from about 3% to about 8% are provided by the claimed invention.

Oral transmucosal bioavailability is typically evaluated following sublingual or buccal administration and GI bioavailability is evaluated following swallowing of drug.

The pharmaceutical composition comprises from about 0.08% to about 2% sufentanil or from about 5 mcg to about 200 mcg of sufentanil.

Oral transmucosal administration results in a Tmax of from about 40 to about 50 minutes with a coefficient of variation of less than 40%.

The dose of sufentanil provides a mean AUC and a mean Cₘₐₓ which is substantially dose proportional when administered to humans by the oral transmucosal route.

The sufentanil composition may comprise from about 1% to 6% HPMC K4M, wherein when subjected to an in vitro dissolution test in a Type II USP dissolution apparatus, at least 70% of the total amount of sufentanil is released within 16 minutes.

Alternatively, the sufentanil composition may comprise from about 1% to 3% HPMC K4M, wherein when subjected to an in vitro dissolution test in a Type II USP dissolution apparatus, at least 70% of the total amount of sufentanil is released within 8 or 12 minutes.

The invention further provides oral transmucosal sufentanil compositions and methods of use in the treatment of pain, for example, acute post-operative pain or breakthrough pain.

The claimed invention further provides a multi-dose dispensing device or SDA comprising an oral transmucosal sufentanil composition as described hereinabove.

### BRIEF DESCRIPTION OF THE DRAWINGS

FIGS. 1A-C are schematic depictions of examples of a multi-dose dispensing device wherein the device is designed to deliver a sufentanil composition to the oral mucosa of a subject. FIG. 1A illustrates an intact drug dispensing device; FIG. 1B shows the reusable head and disposable body and cartridge of the dispensing device; and FIG. 1C is a schematic depiction of a multi-dose dispensing device showing the user interface and a side angle of an exemplary proboscis of the drug dispensing device wherein the proboscis has an S-shape and comprises a shroud.

FIGS. 2A-B provide schematic depictions of an exemplary single dose applicator (SDA) in intact form (FIG. 2B) and as an exploded view showing the component parts (FIG. 2A).

FIGS. 3A-C provide schematic depictions of an exemplary single dose applicator (SDA).

FIGS. 4A-C provide schematic depictions of an exemplary multiple dose dispenser, holding a plurality of SDAs.

FIGS. 5A-C provide schematic depictions of another exemplary multiple dose dispenser, holding a plurality of SDAs (FIGS. 5A and 5C) and showing an SDA after removal from the multiple dose dispenser (FIG. 5B).

FIG. 6 is a graphic depiction of the in vitro dissolution profile of oral transmucosal sufentanil formulations from the Phase 1a and b human clinical studies described in U.S. patent Publication Nos. 20080147044, 20080268023, and 20090131479 and shown in Table 1.

FIG. 7 is a graphic depiction of in vitro dissolution profile of drug formulations prepared with varying amounts of HPMC K4M, as shown in Table 3.

FIGS. 8A and B provide a graphic depiction of observed sufentanil plasma concentration (mean; pg/ml) versus time, following administration to healthy human volunteers using various dosages forms and routes of administration. FIG. 8A shows the pharmacokinetic profile over a period of 6 hours, and FIG. 8B shows the pharmacokinetic profile over a period of 2 hours. (See Example 2.) Treatment A was an IV infusion Sufenta® (5 mcg) and single dose generic triazolam (125 mcg) administered by the oral route; Treatment B was a single tablet containing 15 mcg sufentanil and 200 mcg triazolam administered by the sublingual route; Treatment C was a single tablet containing 15 mcg sufentanil administered by the sublingual route; Treatment D was a single tablet containing 15 mcg sufentanil administered by the buccal route; and Treatment E was three 15 mcg sufentanil tablets administered by the oral route (swallowed). The total 45 mcg dose administered in Treatment E is shown normalized to 15 mcg (mean/3).

### DETAILED DESCRIPTION OF THE INVENTION

### I. Introduction

Oral transmucosal drug compositions and dispensing systems for administration offer numerous advantages over conventional means of drug administration such as the intravenous route (for example, intravenous patient-controlled analgesia or “IV-PCA”), and oral administration. The most important advantage is enhanced safety, with additional advantages being rapid and consistent onset of action, consistent and predictable pharmacokinetics (PK) and pharmacodynamics (PD).

Provided herein are compositions, methods, systems, kits and drug dispensing devices for oral transmucosal administration of sufentanil. Use of small volume drug dosage forms with bioadhesive properties facilitates adherence to the oral mucosa, thus minimizing the risk of swallowing and inefficient delivery due to GI uptake.

The following disclosure provides a description of drug compositions, drug dispensing devices, methods, systems and kits which constitute the invention. The invention is not limited to the specific compositions, devices, methodology, systems, kits or medical conditions described herein, as such may, of course, vary. It is also to be understood that the terminology used herein is for the purpose of describing particular embodiments only, and is not intended to limit the scope of the present invention.

It must be noted that as used herein and in the appended claims, the singular forms “a”, “and”, and “the” include plural references unless the context clearly dictates otherwise. Thus, for example, reference to “a drug formulation” includes a plurality of such formulations and reference to “a drug delivery device” includes systems comprising drug compositions and drug delivery devices for containment, storage and delivery of such drug compositions.

Unless defined otherwise, all technical and scientific terms used herein generally have the same meaning as commonly understood to one of ordinary skill in the art to which this invention belongs. Although any methods, devices and materials similar or equivalent to those described herein can be used in the practice or testing of the invention, the preferred methods, drug delivery devices and materials are now described.

The publications discussed herein are provided solely for their disclosure prior to the filing date of the present application. Nothing herein is to be construed as an admission that the invention is not entitled to antedate such a disclosure by virtue of prior invention.

### II. Definitions

The term “active agent” or “active” may be used interchangeably herein with the term “drug” and is meant to refer to any therapeutically active agent.

The term “adhere” is used herein with reference to a formulation that is in contact with a surface such as a mucosal surface and is retained on the surface without the application of an external force. The term “adhere” is not meant to imply any particular degree of sticking or bonding, nor is it meant to imply any degree of permanency.

The term “analgesic drug” as used herein includes sufentanil or a sufentanil congener, such as alfentanil, fentanyl, lofentanil, carfentanil, remifentanil, trefentanil, or mirfentanil, as well as formulations comprising combinations thereof. Use of the phrase “sufentanil or a congener” is not meant to be limiting to use of, or formulations comprising, only one of these selected opioid compounds. Furthermore, reference to sufentanil alone or to a selected sufentanil congener alone, e.g., reference to “alfentanil”, is understood to be only exemplary of the drugs suitable for delivery according to the methods of the invention, and is not meant to be limiting in any way.

The term “AUC” as used herein means “area under the curve”, and is also referred to as “AUC_{inf}” in a plot of concentration of drug in plasma versus time. AUC is typically given for the time interval zero to infinity, however, clearly plasma drug concentrations cannot be measured ‘to infinity’ for a patient so a mathematical equation is used to estimate the AUC from a limited number of concentration measurements.

AUC_{inf}=AUCₜ+Cₗₐₛₜ/λ_{z} where Cₗₐₛₜ was the last plasma concentration and λz is the terminal disposition rate constant.

In a practical sense, AUC_{inf} represents the total amount of drug absorbed by the body, irrespective of the rate of absorption. This is useful when trying to determine whether two formulations of the same dose release the same dose of drug to the body. The AUC_{inf} of a transmucosal route compared to that of the same drug administered intravenously serves as the basis for a measurement of bioavailability.

The term “bioadhesion” as used herein refers to adhesion to a biological surface including mucosal membranes.

The term “bioavailability” or “F” as used herein means “percent bioavailability” and represents the fraction of drug absorbed from a test formulation as compared to the same drug when administered intravenously. It is calculated from the AUC_{inf} of the test formulation following delivery via the intended route versus the AUC_{inf} for the same drug after intravenous administration. By way of example, the absolute bioavailability of sublingual administration was determined by the following formula:

$$F ( \% ) = \frac{{AUC}_{inf}^{sublingual}}{{AUC}_{inf}^{IV}} \times \frac{{Dose}_{IV}}{{Dose}_{sublingual}}$$

It will understood that bioavailability may also be calculated using AUCt so long as it is clearly indicated how it is calculated. AUCt is measured after the final drug administration without estimating the plasma drug concentration to infinity.

The term “breakthrough pain” as used herein, is a transitory flare of pain of moderate to severe intensity occurring on a background of otherwise controlled pain. “Breakthrough pain” can be intense for short periods of time. A typical breakthrough pain event in cancer patients was reported in a pilot study to range between 15 and 60 minutes.

The term “cartridge” is used herein with reference to a disposable cartridge configured to hold one or more drug dosage forms, typically, about 40 drug dosage forms. The cartridge may comprise a smart cartridge recognition system with a physical keyed feature on the cartridge, a bar code on the cartridge, a magnetic tag on the cartridge, an RFID tag on the cartridge, an electronic microchip on the cartridge, or a combination thereof.

The term “Cₘₐₓ” as used herein means the maximum observed plasma concentration following administration of a drug.

The term “congener” as used herein refers to one of many variants or configurations of a common chemical structure.

As used herein, the term “degradation protectant” refers to any material which protects against degradation of a “drug”, “medication”, or “pharmacologically active agent”. An oxygen scavenger, a desiccant, an anti-oxidant or a combination thereof may serve as a degradation protectant for the sufentanil in a sufentanil-containing solid dosage form.

The term “desiccant” is used herein with reference to a sorbant, in the form of a solid, liquid, or gel which has an affinity for water, and absorbs or adsorbs moisture from it's surrounding, thus controlling the moisture in the immediate environment.

The term “disintegration” means the physical process by which a dosage form breaks down and pertains to the physical integrity of the dosage form alone. This can occur in a number of different ways including breaking into smaller pieces and ultimately, fine and large particulates or, alternatively, eroding from the outside in, until the dosage form is no longer evident by visual examination.

The term “dissolution” as used herein means the process by which the active ingredient is dissolved from a drug formulation in the presence of a solvent, in vitro, or physiological fluids in vivo, e.g., saliva, irrespective of the mechanism of release, diffusion, erosion or combined erosion and diffusion.

The term “dispensing device”, “drug dispensing device”, “dispenser”, “drug dispenser”, “drug dosage dispenser”, “device” and “drug delivery device” are used interchangeably herein and refer to a device that dispenses a drug dosage form. The dispensing device provides for controlled and safe delivery of a pharmaceutically active substance (e.g., an opioid such as sufentanil) formulated as a dosage form. The device may be adapted for storage and/or delivery of a dosage form such as a lozenge, pill, tablet, capsule, membrane, strip, liquid, patch, film, gel, spray or other form.

The term “dispensing end” as used herein with reference to a device means the portion of the device comprising the proboscis and shroud which serves to deliver a drug to the oral mucosa of a subject.

The term “drug”, “medication”, “pharmacologically active agent”, “therapeutic agent” and the like are used interchangeably herein and generally refer to any substance that alters the physiology of an animal and can be effectively administered by the oral transmucosal route.

The term “erosion time” means the time required for a solid dosage form to break down until the dosage form is no longer evident by visual examination.

The terms “formulation” and “drug formulation” as used herein refer to a physical composition containing at least one pharmaceutically active substance, which may be provided in any of a number of forms for delivery to a subject. The drug may be provided to the patient in the form of a lozenge, pill, capsule, membrane, strip, liquid, patch, film, gum, gel, spray or other form.

The term “hydrogel-forming preparation”, means a solid formulation largely devoid of water which upon contact with an aqueous solution, e.g., a bodily fluid, and in particular that of the oral mucosa, absorbs water in such a way that it forms a hydrated gel in situ. The formation of the gel follows unique disintegration (or erosion) kinetics while allowing for release of the therapeutic agent over time. Additionally, the term “hydrogel-forming preparation’ describes a solid formulation largely devoid of water which upon contact with bodily fluids, and in particular those in the oral cavity, transforms into a film that releases the drug. Such films increase the surface area available for drug release and absorption thus enabling faster absorption of the drug.

The term “lock-out feature” is used herein with reference to a feature of the device which provides for a “lock-out time”.

The term “lock-out time” is used herein with reference to the period of time during which a device does not allow drug accessibility, i.e., a dosage form cannot be dispensed during the “lock-out time”. “Lock-out time” may be programmable, a fixed time interval, a predetermined interval, a predetermined variable interval, an interval determined by an algorithm or a variable interval communicated to the device from a remote computer or docking station.

The term “LogP” as used herein means logarithm of the ratio of equilibrium concentrations of un-ionized compound between octanol and water. P also called the “octanol-water partition coefficient” and serves as a means to quantify the hydrophiobicity or lipophilicity of, a chemical characteristic of a given drug.

The term “mucosal membrane” refers generally to any of the mucus-coated biological membranes in the body. Absorption through the mucosal membranes of the oral cavity is of particular interest. Thus, oral mucosal absorption, i.e., buccal, sublingual, gingival and palatal absorption are specifically contemplated.

The term “mucosal-depot” is used herein in its broadest sense to refer to a reservoir or deposit of a pharmaceutically active substance within or just beneath the mucosal membrane.

The term “non-ordered particulate mixture” or “non-ordered mixture” is used herein with reference to a formulation where the mixture is not ordered with respect to the distribution of drug particles over the surface of larger carrier particles. Such ‘non-ordered’ mixing may involve dry mixing of particles in a non-ordered fashion, where there is no requirement with respect to the order of addition/mixing of specific excipients with the drug, bioadhesive material or bioadhesion promoting agent and/or disintegrants. Further in the non-ordered mixing process, there is no limitation on the size of the drug particles. The drug particles may be larger than 25 μm. In addition, a “non-ordered mixture” includes any mixing processes in which the primary carrier particles do not incorporate a disintegrant within. Finally the “non-ordered mixture” may be prepared by any ‘wet mixing’ processes, i.e. processes in which a solvent or non-solvent is added during the mixing process or any mixing process in which the drug is added in a solution or suspension form and wherein the drug particles are not uniformly distributed over the surface of larger carrier particles.

The term “opioid naïve patient” is used herein with reference to a patient who has not received repeated administration of an opioid substance over a period of weeks to months.

The term “opioid tolerant patient” as used herein means a physiological state characterized by a decrease in the effects of an opioid substance (e.g., analgesia, nausea or sedation) with chronic administration. An opioid substance is a drug, hormone, or other chemical substance that has analgesic, sedative and/or narcotic effects similar to those containing opium or its derivatives. If analgesic tolerance develops, the dose of opioid substance must be increased to result in the same level of analgesia. This tolerance may not extend to side effects and side effects may not be well tolerated as the dose is increased.

The terms “oral transmucosal dosage form” and “oral transmucosal formulation” are used herein to refer to a drug formulation which comprises a pharmaceutically active substance, such as sufentanil that is used to deliver the pharmaceutically active substance to the circulation by way of the oral mucosa. The oral transmucosal formulation provides for delivery of the pharmaceutically active substance across the oral mucosa and not via swallowing followed by GI absorption. The dosage form comprises pharmaceutically acceptable excipients as detailed in U.S. patent Publication Nos. 20080147044, 20080268023, and 20090131479, expressly incorporated by reference herein.

The terms “oral transmucosal drug delivery” and “oral transmucosal administration” as used herein refer to drug delivery that occurs substantially via the oral transmucosal route and not via swallowing followed by GI absorption. This includes delivery via buccal, sublingual and gum transmucosal areas. Delivery is typically accomplished by placement of a drug dosage form on the surface of an oral mucosal membrane. Similarly, an “oral transmucosal formulation” is a drug formulation which provides for effective drug delivery via a buccal, sublingual or gum transmucosal membrane.

The term “proboscis” is used interchangeably with the terms “dispensing tip” a “delivery tip”, and refers to a dispensing and/or positioning tip of a drug dispensing device that delivers a drug to the oral mucosa (e.g., the sublingual space).

The term “radio frequency identification device” or “RFID” is used with reference to an automatic identification method, which relies on storing and remotely retrieving data using devices called RFID tags, wherein the RFID tag is applied to, or incorporated into a product, or person for the purpose of identification using radiowaves. Some tags can be read from several meters away and beyond the line of sight of the reader.

The term “replaceable cartridge” or “disposable cartridge” is used with reference to a cartridge for housing drug dosage forms which is typically configured to hold up to about 40 drug dosage forms, wherein the cartridge is designed to be used and discarded.

The term “shroud” is used to describe a partial or complete covering of the dispensing end of the device which protects the delivery port from contact with saliva or other moisture in the oral cavity and forms a barrier between the device, the oral mucosa and tongue. The shroud limits the ability of the tongue or oral mucosa to contact the drug dispensing area, thereby controlling saliva contact and ingress.

The term “subject” includes any subject, generally a mammal (e.g., human, canine, feline, equine, bovine, ungulate etc.), adult or child, in which treatment for a disorder is desired. The terms “subject” and “patient” may be used interchangeably herein.

The term “small volume drug dosage form” or “small volume dosage form” is used herein with reference to a dosage form for oral transmucosal administration that has a mass of from about 1 mg to about 100 mg, from about 2 mg to about 80 mg, from about 3 mg to about 50 mg, from about 4 mg to about 30 mg, from about 3 mg to about 25 mg, from about 3 mg to about 20 mg, from about 3 mg to about 15 mg, from about 3 mg to about 10 mg, from about 5 mg to about 15 mg, from about 5 mg to about 10 mg, or from about 5 mg to about 8 mg. The dosage form may also be characterized in terms of volume. More specifically, the dosage form may have a volume of from about 1 microliter (mcl) to about 100 mcl, from about 2 mcl to about 80 mcl, from about 3 mcl to about 50 mcl, from about 4 mcl to about 30 mcl, from about 3 mcl to about 25 mcl, from about 3 mcl to about 20 mcl, from about 3 mcl to about 15 mcl, from about 3 mcl to about 10 mcl, from about 5 mcl to about 15 mcl, from about 5 mcl to about 10 mcl, or from about 5 mcl to about 8 mcl. The “dosage form” typically has bioadhesive characteristics and may form a hydrogel upon contact with an aqueous solution, such as saliva.

The “dosage form” may be used to deliver any pharmaceutical agent or drug that can be administered by the oral transmucosal route in an amount amenable to administration via the small size of the dosage form, i.e., an amount of drug selected from about 1 mcg to 10 mg, from about 2 mcg to 5 mg, from about 5 mcg to 1 mg, from about 0.01% to about 10%, from about 0.05% to about 5%, from about 0.1% to about 1%, and from about 0.2% to about 2%.

The term “small volume sufentanil-containing drug dosage form” is used herein with reference to a small volume dosage form for oral transmucosal administration that contains a dose of sufentanil selected from about 2 micrograms (mcg) to about 200 mcg of sufentanil, from about 5 mcg to about 100 mcg, from about 10 mcg to about 100 mcg of sufentanil, or from about 20 mcg to about 100 mcg of sufentanil, e.g., 5 mcg, 10 mcg, 15 mcg, 20 mcg, 30 mcg, 40 mcg, 50 mcg, 60 mcg, 70 mcg, 80 mcg, 90 mcg or 100 mcg of sufentanil.

The term “small volume solid drug dosage form” is used herein with reference to a small volume dosage form that is a solid, e.g., a lozenge, a pill, a tablet, a membrane or a strip.

The term “sublingual”, means literally “under the tongue” and refers to administering a drug composition via the mouth in such a way that the pharmaceutically active substance is rapidly absorbed via the blood vessels under the tongue rather than via the digestive tract. Absorption occurs via the highly vascularized sublingual mucosa and allows the pharmaceutically active substance more direct access to the blood circulation, providing for direct systemic administration independent of GI influences.

The term “terminal half-life” or “t_{1/2} [h]” as defined herein is calculated as ln(2)/λ_{z} (defined as the first order terminal rate constant estimated by linear regression of the time versus log concentration curve) and also determined after the final dosing in repeated dose studies.

The term “Tₘₐₓ” as used herein means the time point of maximum observed plasma concentration.

The term “therapeutically effective amount” means an amount of a therapeutic agent, or a rate of delivery of a therapeutic agent (e.g., amount over time), effective to facilitate a desired therapeutic effect, such as pain relief. The precise desired therapeutic effect (e.g., the degree of pain relief, and source of the pain relieved, etc.) will vary according to the condition under treatment, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

The term “transmucosal” delivery of a drug and the like is meant to encompass all forms of delivery across or through a mucosal membrane.

### III. Formulations

Typical formulations for preparation of sufentanil-containing drug dosage forms and methods of making them are described in US Patent Publication Nos. 20070207207 and 20080166404, expressly incorporated by reference herein. An exemplary composition for oral transmucosal administration of sufentanil is bioadhesive and comprises from about 0.04% to about 4% sufentanil, from about 0.08% to about 2.0% sufentanil, from about 0.1% to about 1.5% sufentanil, e.g., about 0.04%, 0.06%, 0.08%, 0.1%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0%, 1.1%, 1.2%, 1.3%, 1.4%, 1.5%, 1.6%, 1.7%, 1.8%, 1.9%, 2.0%, 2.2%, 2.4%, 2.6%, 2.8%, 3.0%, 3.2%, 3.4%, 3.6%, 3.8% or 4% sufentanil.

In general, the formulation is provided as a substantially homogeneous composition which comprises one or more opioids, i.e., sufentanil, one or more of bioadhesives, binders, hydrogel-forming excipients, bulking agents, disintegrating agents, lubricants, and other excipients and factors that affect dissolution time and/or drug stability. Formulations for use in the invention typically comprise: mannitol (Grade 100SD), dicalcium phosphate hydrate and anhydrous (DCP hydrate and anhydrous), hydroxypropyl methyl cellulose (HPMC) grade K4M, croscarmellose sodium, stearic acid, magnesium stearate and butylated hydroxy toluene (BHT). Additionally, the formulation may comprise one or more excipients that facilitate consistent dissolution and/or drug release from the formulation, e.g., a “super disintegrant”.

The drug formulations may be provided as solid dosage forms (e.g., tablets) that are neither effervescent nor do they comprise an ordered mixture of microparticles of drug adhered to the surface of carrier particles, where the carrier particles are substantially larger than the microparticles of drug.

A disintegrant is a substance in a tablet formulation that enables the tablet to break up into smaller fragments which increases the surface area of the tablet particles, thereby increasing the rate of absorption of the active ingredient. A “super disintegrant” has efficient disintegrating properties at relatively low concentrations. A “super disintegrant” is included in a formulation for use in the present invention an amount of from about 1% to about 10%, typically from about 2% to about 4%. Examples of superdisintegrants are crosscarmelose, crospovidone, and sodium starch glycolate, which are crosslinked cellulose, crosslinked polymer, and a crosslinked starch, respectively. BASF is one manufacturer of crospovidone under the trade name of insoluble “Kollidon” grades. Another manufacturer is ISP's “Polyplasdone XL” grade. Crosscarmelose (crosslinked sodium carboxy methylcellulose) is marketed mainly under the brand name of “Ac-Di-Sol” by FMC BioPolymer. See, e.g., http://pharmtech.findpharma.com/pharmtech/article/articleDetail.jsp?id=378399. A further disintegrant useful in the present invention is Primojel®, a sodium starch glycolate USP-NF from DMV International (http://www.metolose.jp/e/pharmaceutical/l-hpc.shtml) produced by cross-linking and carboxymethylation of potato starch. A further binder and a disintegrant useful in solid medicamatns include a low-substituted hydroxypropyl ether of cellulose (“L-HPC”).

HPMC grade K4M with an average viscosity of 4,000 mPas, is generally included in formulations of for use in practicing the invention in an amount of from about 3% to about 12% (w/w), typically from about 3% to about 6%. It will be understood by those of skill that other grades of HPMC may also be used in practicing the present invention, so long as tablet adhesion is not compromised, as indicated in Example 1, Table 3, below.

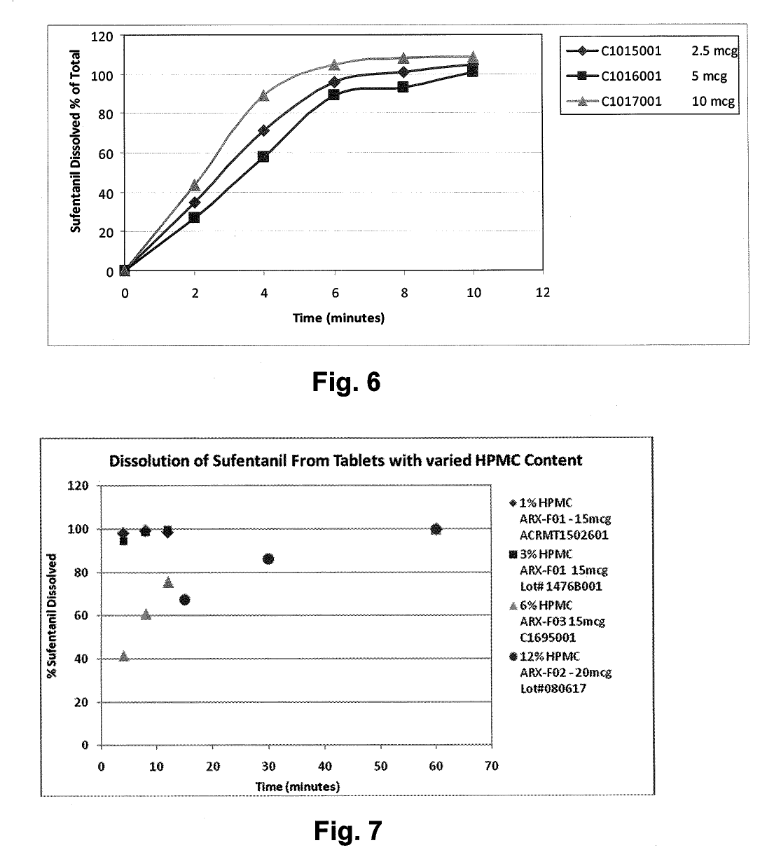

The in vitro dissolution of formulations for oral transmucosal administration of sufentanil have been characterized using a modified USP II dissolution apparatus with LCMS or HPLC detection, as detailed in Example 1, Table 2. When formulations lacking HPMC K4M were subjected to an in vitro dissolution test in a Type II USP dissolution apparatus with LCMS detection, at least 75% of the total amount of sufentanil in oral transmucosal sufentanil tablets was released within 10 minutes (see FIG. 6).

When formulations comprising from about 1% to about 3% HPMC K4M are subjected to an in vitro dissolution test in a Type II USP dissolution apparatus with HPLC detection, at least about 70% of sufentanil is released within 12 minutes, and preferably at least 70% of sufentanil is released within 8 minutes (FIG. 7). When formulations comprising from about 3% to about 6% HPMC K4M are subjected to an in vitro dissolution test in a Type II USP dissolution apparatus with HPLC detection, at least about 70% of sufentanil is released within 16 minutes, and preferably at least 70% of sufentanil is released within 12 minutes (FIG. 7).

Numerous suitable nontoxic pharmaceutically acceptable carriers for use in oral transmucosal compositions can be found in Remington's Pharmaceutical Sciences, 17th Edition, 1985.

It will be understood that the formulation is converted into a dosage form for delivery to a subject using procedures routinely employed by those of skill in the art, such as direct compression, wet granulation, etc. The process for preparation of the dosage form is optimized for each formulation in order to achieve high dose content uniformity.

### IV. Pharmacokinetics (PK) and Pharmacodynamics (PD)

Sufentanil compositions for use in the invention typically have bioadhesive characteristics and may form a hydrogel upon contact with an aqueous solution, i.e., upon contact with the oral mucosa. In another embodiment, the composition is a solid that transforms into a bioadhesive film upon contact with saliva.

Such solid dosage forms typically have a predictable in vivo erosion time, however the erosion time may vary. Following administration of a sufentanil-containing solid dosage form to the surface of an oral mucosal membrane of a subject, the observed in vivo erosion time is from about 2 minutes to about 40 minutes, from about 3 minutes to about 30 minutes, from about 4 minutes to about 25 minutes; from about 4 minutes to about 20 minutes; from about 4 minutes to about 18 minutes; from about 4 minutes to about 16 minutes; from about 4 minutes to about 14 minutes; from about 4 minutes to about 12 minutes; from about 4 minutes to about 10 minutes; from about 5 minutes to about 10 minutes; or from about 5 minutes to about 8 minutes. The subject may be NPO (no oral intake allowed) or non-NPO. In vivo erosion time is defined as the time required for a solid dosage form to break down such that the original dosage form is no longer evident by visual examination.

In general, following oral transmucosal administration of a sufentanil-containing composition, at least 40%, at least 45%, at least 50%, at least 55%, at least 60%, at least 65%, at least 70%, at least 75%, or at least 80%, at least 85%, at least 90%, or at least 95% of the total amount of the pharmaceutically active substance in a sufentanil-containing composition placed on the surface of an oral mucosal membrane of a subject is absorbed via the oral transmucosal route.

A single or repeated oral transmucosal administration of a sufentanil-containing composition to a subject results in a Tₘₐₓ of from about 30 minutes to about 80 minutes; from about 40 minutes to about 70 minutes; from about 40 minutes to about 60 minutes; from about 40 minutes to about 50 minutes; or from about 45 minutes to about 55 minutes.

A single or repeated oral transmucosal administration of a sufentanil-containing composition to a subject results in a Tₘₐₓ with a coefficient of variation of from about 30% to about 50%; from about 35% to about 50%; from about 35% to about 45%; from about 40% to about 50%; less than about 45%; less than about 42%; less than about 40%; less than about 38%; less than about 36%; less than about 34%; less than about 32%; or less than about 30%.

A single or repeated oral transmucosal administration of a sufentanil-containing composition to a subject results in a Cmax with a coefficient of variation of from about 30% to about 60%; from about 35% to about 55%; from about 35% to about 50%; from about 35% to about 45%; from about 40% to about 50%; less than about 45%; less than about 42%; less than about 40%; less than about 38%; less than about 36%; less than about 34%; less than about 32%; or less than about 30%.

A single or repeated oral transmucosal administration of a sufentanil-containing composition to a subject results in a results in a bioavailability of from about 50% to about 90%; about 50% to about 80%; from about 55% to about 75%; from about 55% to about 70%; from about 55% to about 65%; or greater than about 50%; greater than about 55%; greater than about 60%; greater than about 65%; greater than about 70%; greater than about 75%; greater than about 80%; greater than about 85%; greater than about 90%; or greater than about 95%. Oral transmucosal administration is typically sublingual or buccal administration.

A single or repeated oral transmucosal administration of a sufentanil-containing composition to a subject results in a bioavailability with a coefficient of variation of from about 30% to about 50%; from about 35% to about 50%; from about 35% to about 45%; from about 40% to about 50%; less than about 45%; less than about 42%; less than about 40%; less than about 38%; less than about 36%; less than about 34%; less than about 32%; or less than about 30%.

A single or repeated oral administration of a sufentanil-containing composition, e.g., by swallowing, results in a gastrointestinal (GI) bioavailability from about 2% to about 12%; from about 3% to about 8%; from about 3% to about 7%; from about 4% to about 7%; from about 4% to about 6%; typically less than about 10%; less than about 9%; less than about 8%; or less than about 7%.

A single or repeated oral transmucosal administration of a sufentanil-containing composition to a subject results in an onset of pain relief in from about 5 minutes to about 30 minutes, from about 5 minutes to about 20 minutes, from about 5 minutes to about 15 minutes, or from about 5 minutes to about 10 minutes. More specifically, a single or repeated oral transmucosal administration of a sufentanil-containing composition to a subject results in an onset of pain relief in less than 30 minutes, typically in less than 15 minutes, preferably in less than 10 minutes and more preferably in less than 5 minutes.

### V. Oral Transmucosal Dosage Forms

Oral transmucosal drug delivery is simple and non-invasive. A sufentanil formulation for oral transmucosal delivery may be solid or non-solid.

Oral transmucosal delivery of pharmaceutically active substances may be achieved using solid dosage forms such as lozenges or tablets, however, liquids, sprays, gels, gums, powders, and films and the like may also be used. As will be understood by those of skill in the art, any dosage form may be used in practicing the claimed invention so long as the composition provides for efficacious delivery of sufentanil via the oral mucosal route and a consistent plasma level within the therapeutic window. For lipophilic drugs, such as sufentanil, oral transmucosal delivery has a shorter onset time (i.e., the time from administration to therapeutic effect) than does oral GI delivery and provides better bioavailability and more consistent pharmacokinetics.

The compositions claimed herein provide for substantial delivery of the drug in the composition via the oral mucosa. In contrast to traditional oral dosage forms and other oral transmucosal dosage forms, this results in minimal or no drug delivery via the gastrointestinal (GI) tract.

Typically, the composition is adapted to adhere to the oral mucosa during the period of drug delivery, and remain adhered to the oral mucosal membrane until most or all of the drug has been delivered from the composition to the oral mucosa.

Exemplary dosage forms comprising sufentanil-containing oral transmucosal formulations less have a mass of less than 100 mg, 90 mg, 80 mg, 70 mg, 60 mg, 50 mg, 40 mg, 30 mg, 29 mg, 28 mg, 27 mg, 26 mg, 25 mg, 24 mg, 23 mg, 22 mg, 21 mg, 20 mg, 19 mg, 18 mg, 17 mg, 16 mg, 15 mg, 14 mg, 13 mg, 12 mg, 11 mg, 10 mg, 9 mg, 8 mg, 7 mg, 6 mg, 5 mg, 4 mg, 3 mg or 2 mg; and a mass greater than 1 mg.

More specifically, the dosage forms have a mass of from about 1 mg to about 100 mg, from about 2 mg to about 80 mg, from about 3 mg to about 50 mg, from about 4 mg to about 30 mg, from about 3 mg to about 25 mg, from about 3 mg to about 20 mg, from about 3 mg to about 15 mg, from about 3 mg to about 10 mg, from about 5 mg to about 15 mg, from about 5 mg to about 10 mg, or from about 5 mg to about 8 mg.

Exemplary dosage forms may also be characterized in terms of volume. Accordingly, a dosage form for use in practicing the invention may have a volume of less than 100 mcl, 90 mcl, 80 mcl, 70 mcl, 60 mcl, 50 mcl, 40 mcl, 30 mcl, 29 mcl, 28 mcl, 27 mcl, 26 mcl, 25 mcl, 24 mcl, 23 mcl, 22 mcl, 21 mcl, 20 mcl, 19 mcl, 18 mcl, 17 mcl, 16 mcl, 15 mcl, 14 mcl, 13 mcl, 12 mcl, 11 mcl, 10 mcl, 9 mcl, 8 mcl, 7 mcl, 6 mcl, 5 mcl, 4 mcl, 3 mcl, or 2 mcl, and a volume greater than 1 mcl.

More specifically, the dosage form may have a volume of from about 1 mcl to about 100 mcl, from about 2 mcl to about 80 mcl, from about 3 mcl to about 50 mcl, from about 4 mcl to about 30 mcl, from about 3 mcl to about 25 mcl, from about 3 mcl to about 20 mcl, from about 3 mcl to about 15 mcl, from about 3 mcl to about 10 mcl, from about 5 mcl to about 15 mcl, from about 5 mcl to about 10 mcl, or from about 5 mcl to about 8 mcl.

Solid tablets for use in practicing the invention have an average thickness of about 0.85 mm, typically from about 0.70 mm to about 1.0 mm, from about 0.79 mm to about 0.91 mm, or from about 0.68 mm to about 0.87 mm. Such solid tablets are further characterized by an average diameter of about 3.05 mm, typically from about 3.02 mm to about 3.2 mm; and an average weight of about 5.8 mg, typically from about 5.4 mg to about 6.2 mg. More specifically, a solid tablet for use in practicing the invention has appropriate dimensions such that the tablet adheres to the surface of (on top of) an oral mucosal membrane and is not swallowed during the period of drug delivery.

A solid tablet may be further characterized by the “Aspect Ratio”.

Aspect Ratio=diameter/thickness

Based on this equation: a tablet with a thickness of 0.85 mm and a diameter of 3 mm, the aspect ratio is about 3.5; for a 6 mm diameter tablet, the aspect ratio is about 7.0; for a 0.5 inch (12.5 mm) diameter tablet, the aspect ratio is about 15; and for a 1 inch (25 mm) diameter tablet, the aspect ratio is about 29.

The dosage forms may have essentially any shape, examples of which include a round disc with a flat, concave, or convex face, an ellipsoid shape, a spherical shape, a polygon with three or more edges and flat, concave, or convex faces. The dosage forms may be symmetrical or asymmetrical, and may have features or geometries that allow for controlled, convenient, and easy storage, handling, packaging or dosing.

### VI. Packaging/Prevention or Inhibition of Oxidative Degradation

Many drugs are susceptible to oxidative degradation. In particular, this can be a problem when the drug is present as a low percentage of the overall drug formulation. In order to minimize or eliminate the presence of impurities in a drug formulation comprising an oxidation-susceptible active drug, and a dosage form made from such a formulation, e.g., a solid sufentanil dosage form, preservatives and antioxidants are often employed in the formulation to address this problem. In most cases, this is sufficient to minimize or eliminate the generation of oxidative degradation products.

Solid dosage forms comprising sufentanil may be protected against oxidative degradation by inclusion of an oxygen scavenger in the packaging of the drug dosage form, as described for example in PCT Application Serial No., PCT/US09/64232, expressly incorporated by reference herein. Using this process, solid sufentanil dosage forms are produced and stored under conditions wherein the active drug is protected from oxidative degradation, thus facilitating storage of the drug for extended periods of time.

Additional protection against oxygen exposure may be afforded by employment of packaging techniques designed to minimize exposure of the active drug to oxygen and/or moisture. Exemplary packaging techniques include use of primary packaging wherein more than one oxygen scavenger material is employed alone or in combination with use of a desiccant.

Suitable oxygen scavengers include any organic or inorganic material that can absorb oxygen, for example, iron oxide powders, ferrous salts such as ferrous sulfate or ferrous chloride, sulfites, bisulfites, reducing sulfur compounds such as dithionite, ascorbic acid and/or their salts, erythorbic acid and/or their salts, reducing organic compounds such as catechol and hydroquinone, butylated hydroxytoluene (BHT), butylated hydroxyanisole (BHA). See, e.g. U.S. Patent Publication Nos. 20060076536, 20070084144 and 20060260967.

A number of oxygen scavengers and moisture absorbents are commercially available and may be purchased alone or in packages, e.g., StabilOx® (Multisorb Technologies), cyclohexene methyl acrylated (EMCM) polymer (Chevron-Phillips Chemical Company) or Ciba's Specialty Chemical's SHELFPLUS™).

An oxygen scavenger may be included in the packaging in the form of pellets, canisters, packets, capsules, powders, solid materials, tablets, as part of the packaging material itself or in a foil pouch.

### VII. Sufentanil

Opioids are widely used for the treatment of pain, and are generally delivered intravenously, orally, epidurally, transdermally, rectally and intramuscularly. Morphine and its analogues are commonly delivered intravenously and are effective against severe, chronic and acute pain. However, they can also have severe respiratory depressive effects if not used appropriately and also suffer from a high abuse potential. The predominant cause of morbidity and mortality from pure opioid overdoses is due to respiratory complications.

Sufentanil (N-[(4-(Methoxymethyl-1-(2-(2-thienyl)ethyl)-4-piperidinyl)]-N-phenylpropanamide), is used as a primary anesthetic, to produce balanced general anesthesia in cardiac surgery, for epidural administration during labor and delivery and has been administered experimentally in both intranasal and liquid oral formulations. A commercial form of sufentanil used for IV delivery is the SUFENTA FORTE® formulation. This liquid formulation contains 0.075 mg/ml sufentanil citrate (equivalent to 0.05 mg of sufentanil base) and 9.0 mg/ml sodium chloride in water. It has a plasma elimination half-life of 148 minutes, and 80% of the administered dose is excreted in 24 hours.

The use of sufentanil clinically has predominantly been limited to IV administration in operating rooms or intensive care units.

A number of drug formulations which contain fentanyl are currently available for treatment of pain. It has been reported that administration of fentanyl and other opiate agonists has resulted in potentially deleterious side effects including respiratory depression, nausea, vomiting and constipation.

There is evidence which suggests that sufentanil may result in less respiratory depression at clinical doses as compared to fentanyl and other opioids (Ved et al., 1989; Bailey et al., 1990; Conti et al., 2004).

Sufentanil and fentanyl have many similarities as potent mu-opioid receptor agonists, however, they have been shown to differ in many key ways. Multiple studies have demonstrated sufentanil to be in the range of 7-24 times more potent than fentanyl (SUFENTA® package insert; Paix A, et al. Pain, 63:263-69, 1995; Reynolds L, et al., Pain, 110:182-188, 2004). Therefore, sufentanil may be administered using a smaller dose, avoiding the increased saliva response of a larger dosage form and thereby minimizing the amount of drug that is swallowed resulting in minimal GI uptake.

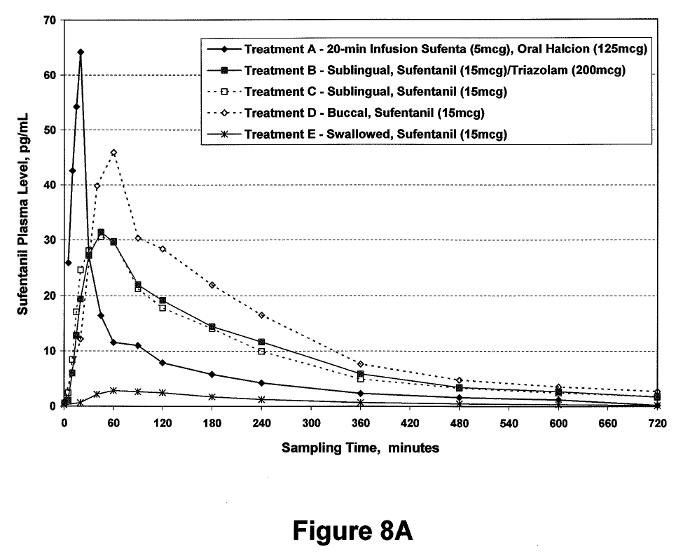

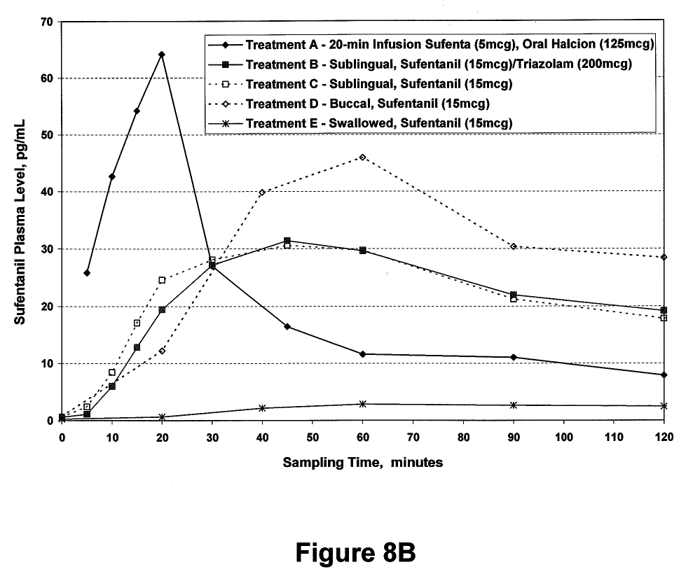

Given that fentanyl has a GI bioavailability of 30-40%, swallowed drug can contribute to plasma levels of fentanyl to a significant degree and result in an erratic Cₘₐₓ and Tₘₐₓ. In contrast, the GI bioavailability of sufentanil is from about 2% to about 8%; from about 3% to about 7%; from about 4% to about 7%; from about 4% to about 6%; less than about 10%; less than about 9%; less than about 8%; or less than about 7% (FIGS. 8A and B), and therefore swallowed sufentanil will not contribute to plasma levels to any significant extent.

Further, the lipid solubility (octanol-water partition coefficient) of sufentanil (1778:1) is greater than fentanyl (816:1) (van den Hoogen and Colpaert, Anesthes. 66:186-194, 1987). Sufentanil also displays increased protein binding (91-93%) compared with fentanyl (80-85%) (SUFENTA® and Actiq® package inserts, respectively). Sufentanil has a pKa of 8.01, whereas the pKa of fentanyl is 8.43 (Paradis et al., Therapeutic Drug Monitoring, 24:768-74, 2002). These differences can affect various pharmacokinetic parameters, for example, sufentanil has been shown to have a faster onset of action and faster recovery time than fentanyl (Sanford et al., Anesthesia and Analgesia, 65:259-66, 1986). As compared to fentanyl, use of sufentanil can result in more rapid pain relief with the ability to titrate the effect and avoid overdosing.

Sufentanil Compositions

The active agent in the claimed formulations is sufentanil or a derivative thereof, alone or in combination with a sufentanil congener such as alfentanil, fentanyl, lofentanil, carfentanil, remifentanil, trefentanil, or mirfentanil. In a preferred embodiment, sufentanil alone is the active agent. Sufentanil may be provided in any of a number of formulations as sufentanil citrate, sufentanil base, or a combination thereof. It will be understood that the term, “sufentanil” is used herein with reference to sufentanil citrate, sufentanil base or a derivative thereof.

A sufentanil drug formulation may contain from about 1 mcg to about 200 mcg of sufentanil per dose for sublingual delivery, alone or combination with one or more other therapeutic agents or drugs.

Exemplary drug formulations for administration to children (pediatric patients) contain from about 1 mcg to about 100 mcg, from about 2 mcg to about 80 mcg of sufentanil, from about 4 mcg to about 60 mcg, from about 5 mcg to about 40 mcg of sufentanil, or from about 10 mcg to about 30 mcg of sufentanil per dose. For example, an oral transmucosal dose of sufentanil for administration to children may contain about 1 mcg, 2 mcg, 4 mcg, 5 mcg, 6 mcg, 8 mcg, 10 mcg, 15 mcg, 20 mcg, 40 mcg, 60 mcg, 80 mcg or 100 mcg of sufentanil.

Exemplary drug formulations for administration to adults contain from about 2 mcg to about 200 mcg of sufentanil per dose. For example, an oral transmucosal dose of sufentanil for administration to adults contains a dose of sufentanil selected from about 5 mcg to about 100 mcg, from about 10 mcg to about 100 mcg of sufentanil, or from about 20 mcg to about 100 mcg of sufentanil, e.g., 5 mcg, 10 mcg, 15 mcg, 20 mcg, 30 mcg, 40 mcg, 50 mcg, 60 mcg, 70 mcg, 80 mcg, 90 mcg or 100 mcg of sufentanil.

Preferably, an individual dose of sufentanil comprises from about 5 to about 100 mcg of sufentanil, e.g., 5 mcg, 10 mcg, 15 mcg, 20 mcg, 30 mcg, 40 mcg, 50 mcg, 60 mcg, 70 mcg, 80 mcg, 90 mcg or 100 mcg of sufentanil.

As will be understood by those of skill in the art, the dose will be on the low end of the range for children and the high end of the range for adults dependent upon body mass, in particular when administered long term to opioid-tolerant adults.

In various embodiments, the claimed formulations provide effective pain relief in all types of patients including children, adults of all ages who are opioid tolerant or naïve and non-human mammals. The invention finds utility in both the inpatient and outpatient setting and in the field.

Congeners of Sufentanil

Congeners of sufentanil also find use in the compositions, methods and systems described herein, examples of which include alfentanil, lofentanil, carfentanil, remifentanil, trefentanil or mirfentanil.

In certain embodiments, formulations of the invention for administration of a sufentanil congener selected from the group consisting of alfentanil, fentanyl, lofentanil, carfentanil, remifentanil, trefentanil, and mirfentanil comprise from about 0.2% to about 20%, from about 0.5% to about 10%, from about 0.75% to about 7.5%, or from about 1% to about 5% of the pharmaceutically active drug.

Individual formulations comprising alfentanil, fentanyl, lofentanil, carfentanil, remifentanil, trefentanil, or mirfentanil typically have an amount of drug selected from about 10 mcg to about 1000 mcg, from about 25 mcg to about 750 mcg, or from about 50 mcg to about 500 mcg of the pharmaceutically active drug. The percentage of active drug will vary dependent upon the size of the dosage form and nature of the active ingredient(s), optimized to obtain maximal delivery via the oral transmucosal route. In some aspects of the invention, more than one active ingredient may be included in a formulation or single dosage form.

### VIII. Treatment of Pain

Using current treatment methods, pain control is attempted using a number of interventions, which generally include: patient-controlled analgesia (PCA), continuous epidural infusion (CEI), other acute pain control, palliative care pain control, and home health patient pain control. These methods meet with varying degrees of success with respect to duration of control, ease of treatment and safety versus side effects.

The need for rapid treatment of acute pain occurs in many different clinical situations, including post-operative recuperation, rheumatoid arthritis, failed back, end-stage cancer (i.e., breakthrough pain). Post-operatively, for example, patients suffer from severe pain for the first few days followed by days of mild to moderate levels of pain.

The most common analgesic used to treat moderate to severe post-operative pain is IV morphine. This is either delivered on an “as needed” basis to the patient by a nurse using IV injection or a morphine syringe placed in a PCA pump which allows the patient to self-administer the opioid by pressing a button outside a lock-out window. Other opioids, such as hydromorphone and fentanyl may also be administered in this manner. In spite of the wide use and advantages of intravenous (IV) patient-controlled analgesia (PCA), its inherent complexity associated with ordering, dispensing, set-up, programming, and administration have resulted in many analgesia related post-operative medication errors (Grass J. Anesth Analg 2005; 101:S44-S61; Hankin C, et al., Am J Health-Syst Pharm 2007; 64:1492-1499; Hicks R. et al., Jt Comm J Qual Patient Saf 2008; 34(12):734-742; and Meissner B, et al., Hospital Pharmacy 2009; 44(4):312-324). Errors utilizing IV PCA have been reported across all phases of the medication-use process, but human factors, such as programming or administering the wrong dose, are among the most common and serious type of errors. Among the human factors, operator errors (81% involving pump programming errors) are the most concerning since they account for the majority of errors that cause harm to patients. Approximately 5% of operator errors reported in the MAUDE database resulted in patient deaths (Hankin et al., 2007).

Treatment of acute pain is also necessary for patients in an outpatient setting. For example, many patients suffer from chronic pain and require the use of opioids on a regular basis to treat their pain. While they may take a long-acting oral or transdermal opioid preparation to treat their chronic underlying pain level, they often need short-acting potent opioids to treat their severe breakthrough pain.

Treatment of acute pain is also necessary “in the field” under highly sub-optimal conditions. Paramedics or military medics often are required to treat severe acute pain in non-sterile situations, where needles used for IV or IM administration can result in unintended needle sticks, risk of infection, etc. Oral opioid tablets often take 60 minutes to provide relief which is too long for someone in severe pain.

Patients suffering from chronic painful conditions can also have intermittent or breakthrough exacerbations of their pain, despite relatively stable and adequately controlled background pain. Such patients often require acute use of fast-acting breakthrough treatment in addition to their use of slow-onset time-release opioids for their baseline chronic pain. The successful management of breakthrough pain requires supplemental analgesia, known as rescue medication. Preferably, the rescue medication should have a rapid onset, good efficacy, relatively short duration of action, and minimal side effects. The duration of a breakthrough pain event in cancer patients was reported in a pilot study to range between 15 and 60 minutes (Zeppetella G., Eur J Cancer Care (Engl) 2009 July; 18(4):331-7).

In a number of clinical settings, there is clearly a need for improved means to administer a drug that produces effective pain relief in a manner that is titratable, safe, non-invasive and convenient and that provides relief from acute, severe breakthrough or intermittent pain over an appropriate period of time. In addition, patient use and lack of abuse and/or diversion must be easily tracked by the physician.

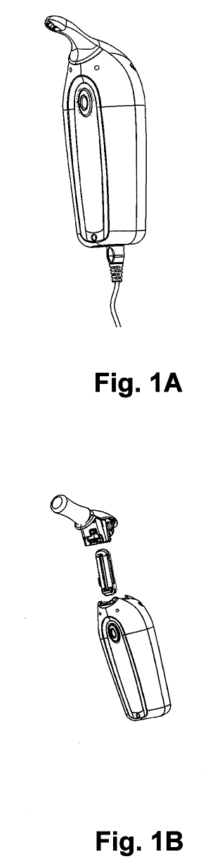

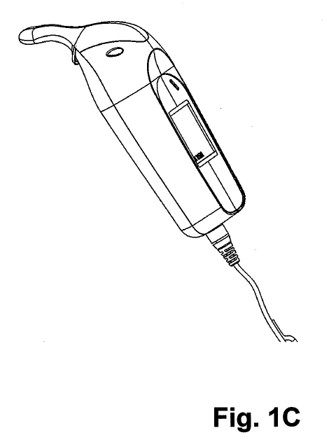

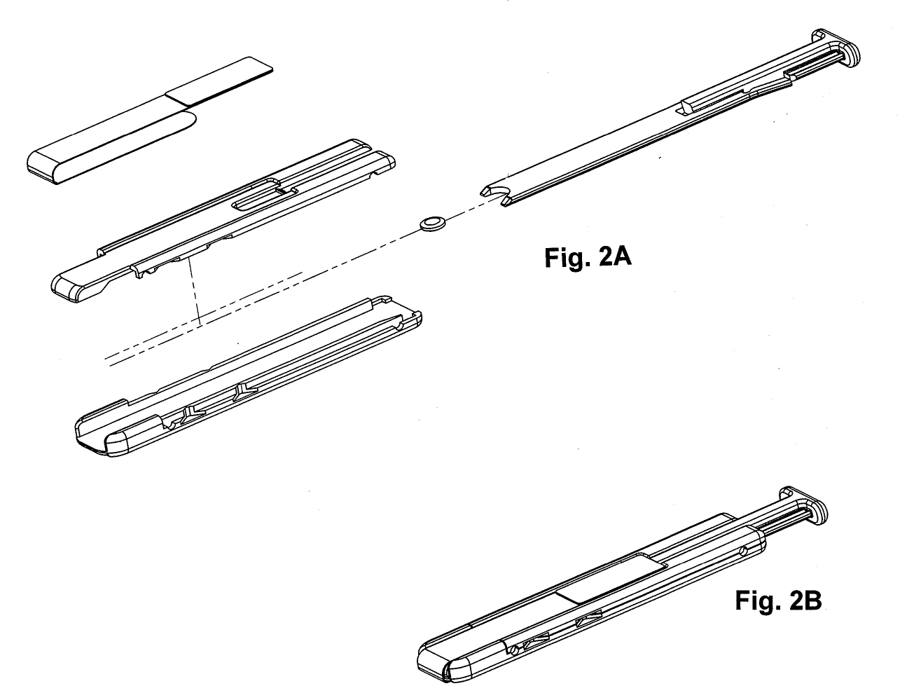

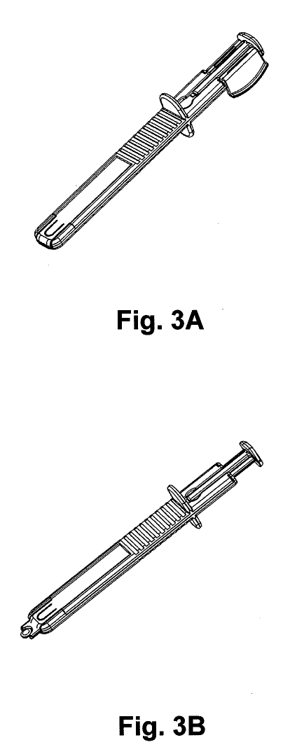

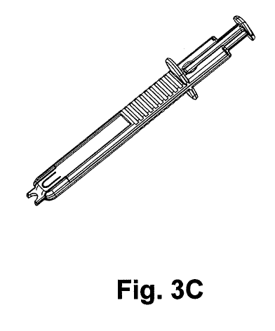

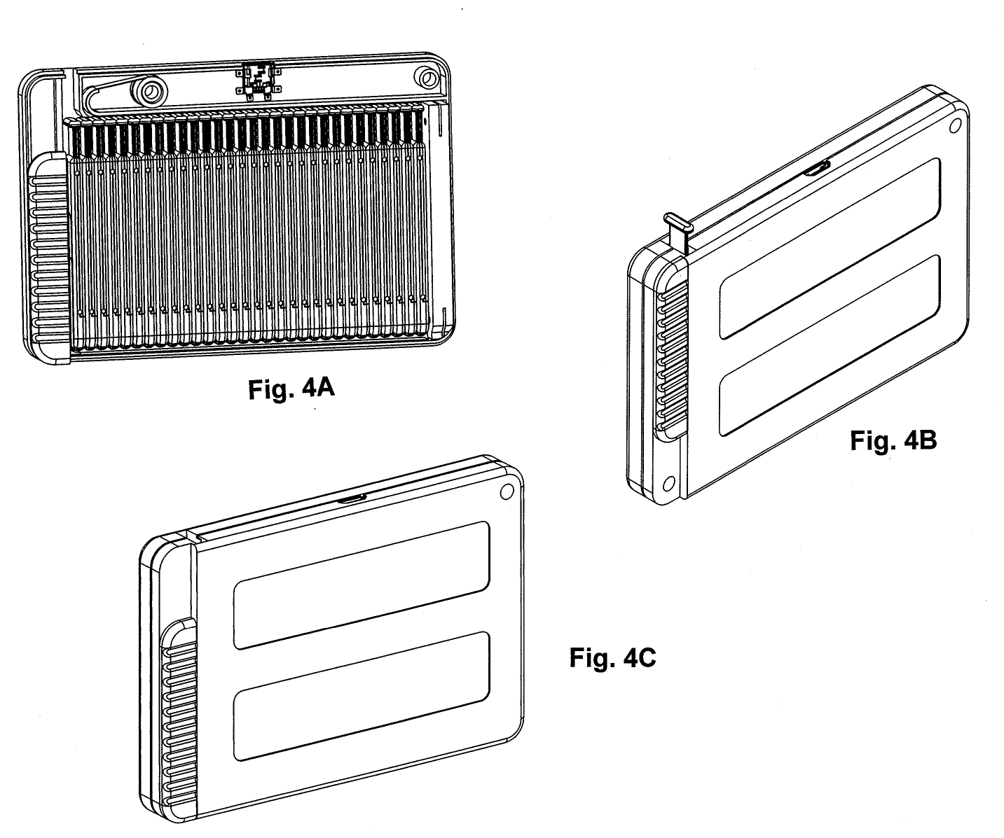

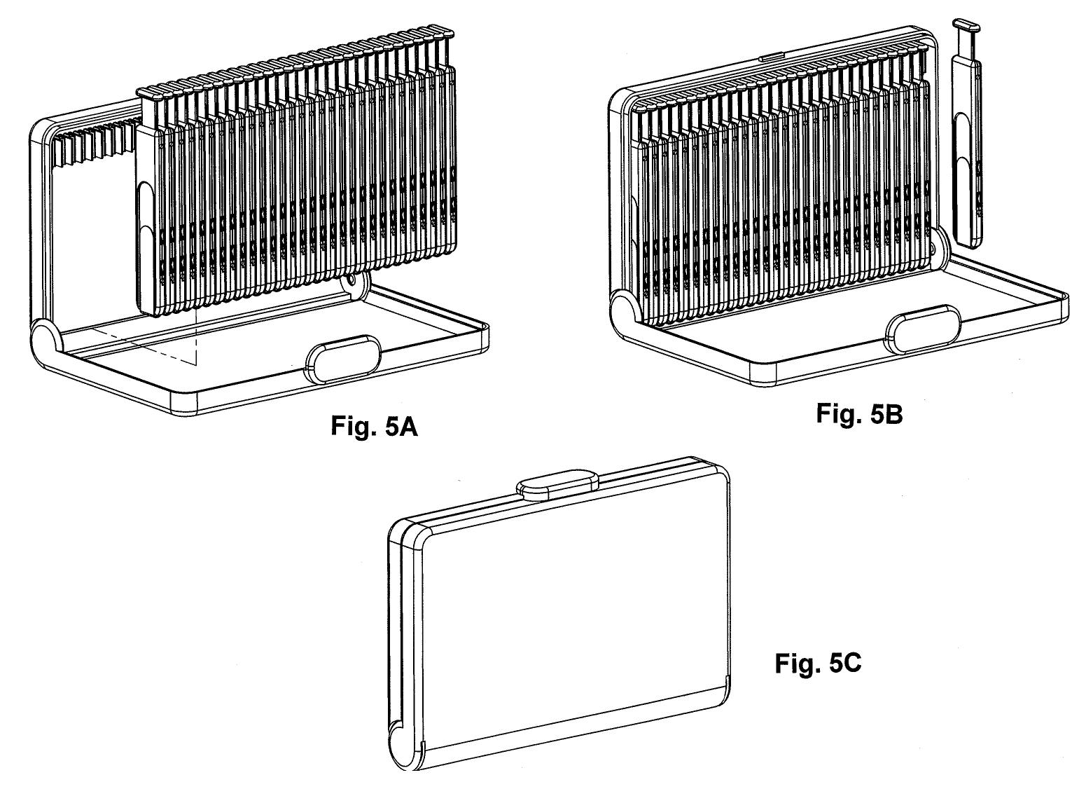

The claimed compositions, methods and systems rely on administration of sufentanil for the treatment of acute pain (i.e. post-operative pain), intermittent pain or breakthrough pain, using a dispensing device that may include features such as lock-out, a means for user identification prior to drug administration, a means for dose tracking and a means to protect the sufentanil compositions stored in the dispensing device. (See, e.g., FIGS. 1A-C).

### IX. In Vivo Human Studies

Provided herein is pharmacokinetic data obtained in humans where sufentanil was administered via the oral transmucosal route using small volume solid dosage forms. Human clinical studies were performed using healthy human volunteers. The study detailed in Example 2 was performed with 12 subjects using 5 treatment groups, as follows:

Treatment A: IV infusion Sufenta® (5 mcg) and single oral dose generic Triazolam (125 mcg);

Treatment B: a single sublingual tablet containing 15 mcg sufentanil and 200 mcg triazolam administered by the sublingual route;

Treatment C: a single tablet containing 15 mcg sufentanil administered by the sublingual route;

Treatment D: a single tablet containing 15 mcg sufentanil administered by the buccal route; and

Treatment E: three 15 mcg sufentanil tablets administered orally (swallowed).

Also provided herein is a summary of Phase 2 human clinical trials wherein sufentanil was administered via the oral transmucosal route for the treatment of acute post-operative pain following elective unilateral knee replacement surgery including clinical efficacy, safety, and tolerability and device functionality (Example 3) or abdominal surgery including clinical efficacy, safety, and tolerability (Example 4). The results of another Phase 2 human clinical trial wherein sufentanil was administered via the oral transmucosal route for the treatment of breakthrough pain in cancer patients are summarized in Example 5.

### X. Utility of Oral Transmucosal Sufentanil Compositions

The claimed sufentanil compositions, methods and systems find utility in delivery of sufentanil via the oral transmucosal route for the treatment of pain. The claimed sufentanil compositions provide for relatively high oral transmucosal bioavailability as compared to GI bioavailability, low variability in Tₘₐₓ, low variability in Cₘₐₓ, low variability in AUC, and little to no drug delivery by the GI route. When administered by the oral transmucosal route, the compositions provide for prolonged plasma levels within the therapeutic window.

In one exemplary embodiment described in detail herein, the sufentanil compositions find utility in treating a subject suffering from pain that may be associated with any of a variety of identifiable or unidentifiable etiologies. In this embodiment, the sufentanil compositions find utility in suppression or mitigation of pain. The term “treatment” or “management” of pain is used here to generally describe regression, suppression, or mitigation of pain so as to make the subject more comfortable, as determined for example by pain score.

The claimed compositions find utility in the treatment of both opioid naïve patients and opioid tolerant patients.

When sufentanil compositions are used for the treatment of pain, the claimed methods and systems find utility in administration of drugs to pediatric and adult populations and in treatment of human and non-human mammals.

Application of the claimed methods and systems is not limited to any particular therapeutic indication. As such, the claimed sufentanil compositions find utility in administration of sufentanil to pediatric and adult subjects and in the treatment of human and non-human mammals.

The sufentanil compositions find utility in pediatric applications, since the comfortable and secure nature of the sufentanil composition allows children to readily accept this mode of therapy and will reliably deliver drug transmucosally. Specific examples include, but are not limited to, treatment of pediatric acute pain when IV access is not available or inconvenient, treatment of pediatric asthma when the child is not able to use an inhaled route of administration effectively, treatment of nausea when a child can not or will not swallow a pill, pre-procedural sedation when a child is NPO (no oral intake allowed) or a more rapid onset is required.

The sufentanil compositions find further utility in veterinary applications. Specific examples include, but are not limited to, any treatment of an acute condition for which IV administration is not readily available or inconvenient, such as pain relief, anxiety/stress relief, pre-procedural sedation, etc.

### XI. Dispensing Devices

Single and multi-dose dispensing devices, methods and systems for oral transmucosal administration of sufentanil compositions are provided. The dispensing devices are handheld and portable and are effective for delivery and monitoring of drug administration. The multi-dose dispensing device may be used to deliver sufentanil, wherein the amount of drug delivered, corresponding efficacy and safety are enhanced over currently available systems. The system may have one or more features that provide for improved safety and ease of use over currently available systems including a security feature that prevents unauthorized access to the stored drugs, a dosing lock-out feature, a means for identifying an individual user for controlled drug access, a dose counting feature, a memory means for retaining information about dose delivery, and an interface for bidirectional exchange of information with a user, a drug cartridge, or another device such as a computer.

The invention is not limited to specific devices, systems, methodology or dosage forms, as these may, of course, vary. It is also to be understood that the terminology used herein is for the purpose of describing particular embodiments only, and is not intended to limit the scope of the present invention.

Dispensing devices for use in practicing the invention are described in detail U.S. Patent Publication Nos. 20070186923 and 20080164275, and U.S. patent application Ser. No. 12/724,634, expressly incorporated by reference herein.

The dispensing device may employ any means of user identification, including fingerprint identification, RFID detection with the use of an active or passive RFID tag on bracelet, necklace, clip, belt, strap, adhesive patch, e.g., a thumb or finger patch, implant, or means of locating and affixing a tag, retinal identification, DNA identification, voice recognition, password or code entry, physical key, electronic or magnetic key, personal area network identification using the human body or clothing as a data or signal conduit, optical scanner or face recognition, sonic, subsonic or ultrasonic identification, or any other means of identifying an individual and verifying their identity.

The dispensing device may provide for lock out, requiring the patient to communicate with the physician or other authorized care giver to unlock the device prior to drug administration. In this way the device and dock provide for safe drug administration due to greater physician oversight and care management.

The timed lock-out period is typically from about 5 minutes to about 30 minutes, from about 10 minutes to about 25 minutes, from about 15 minutes to about 20 minutes, e.g., 20 minutes. In particular cases, the lock-out time is set at a fixed interval between 5 and 60 minutes, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 minutes.

The invention also provides disposable applicators, e.g., a SDA, for delivering sufentanil to the oral mucosa of a subject. The sufentanil composition is provided in a child-resistant drug dispensing device and/or packaging. The sufentanil composition may be self-administered or alternatively, the sufentanil composition is administered with assistance with or without a device.

In one embodiment, SDA is used to deliver a sufentanil composition, provided as a solid tablet, a liquid capsule, a gel capsule, a liquid, a gel, a powder, a film, a strip, a ribbon, a spray, a mist, a patch, or any other suitable drug sufentanil composition.

The SDA may contain the sufentanil composition within, may have the sufentanil composition attached or affixed to it, may have the sufentanil composition dissolved in it, and may afford a seal against moisture, humidity, and light. The SDA may be manually manipulated by a patient, healthcare provider, or other user to place the sufentanil composition in the proper location for drug delivery.

In practicing the invention, a single- or multiple-dose applicator or drug dispensing device may be used to deliver tablets or other sufentanil compositions into the hand, mouth, under the tongue, or to other locations appropriate for specific drug delivery needs.

The sufentanil compositions inside the dispensing device remain dry prior to dispensing, at which point a single dose is dispensed from the device into the mouth, e.g., to the surface of an oral mucosal membrane wherein a patient's saliva will facilitate drug dissolution and delivery.

The SDA may be provided as a pair of forceps, a syringe, a stick or rod, a straw, a pad, a capsule, a cup, a spoon, a strip, a tube, an applicator, a dropper, a patch, an adhesive pad, an adhesive film, a sprayer, an atomizer, or any other form suitable for the application of a single drug dose to the oral mucosa of a subject. As will be understood by one of skill in the art, the SDA design may vary, so long as it is effective to place the drug in the desired location on the surface of an oral mucosal membrane, in a manner that preserves the integrity of the drug dosage form in the dispensing process. After use, the SDA is disposed of, so as to eliminate the risk of contaminating the drug dispensing device with saliva, or other contaminants.

In one approach, a plurality of SDAs is provided as a series of individual SDAs attached by the backing or housed in multiple dose dispenser (MDD), also referred to as a multiple single dose dispenser (MSD).

In a related embodiment, the drug dispensing device contains a plurality of SDAs, in a cartridge or individually packaged, and may dispense a single SDA containing a single drug dose for use by a patient, healthcare provider, or other user. The drug dispensing device may dispense single SDAs in the same way and with the same features as would be advantageous for the dispensing of single drug dosage forms described herein.

In yet another embodiment the multiple dose applicator is a device which comprises one or more drug doses or SDAs, a portable power means, like a battery, a printed circuit board, a data connectivity means, and a user interface. In this embodiment the drug dispensing device may include the ability to perform one or more of the following functions: record drug dosage dispensing history, check user identification, allow the dosage history to be transferred to another device, computer or network, and/or provide a lockout period between dose dispenses. SDAs and MDDs are described in detail U.S. Patent Publication Nos. 20070186923, 20080164275 and 20090048237, and U.S. patent application Ser. Nos. 12/275,485, 12/724,634 and 12/580,930, expressly incorporated by reference herein.

The following examples are provided to illustrate the invention and are not intended to limit any aspect of the invention as set forth above or in the claims below.

### EXAMPLES

### Example 1

### In Vitro Dissolution Testing of Sufentanil Formulations

The in vitro dissolution of sufentanil formulations was evaluated using a USP Type II dissolution apparatus. The dissolution test was conducted under the following conditions.

Apparatus: USP II apparatus—paddles at 15 mm height

Dissolution Media: 10 mM Tris-Acetic Acid buffer at pH 7.4

Dissolution Volume: 500 mL

Temperature: 37 deg

Speed: 50 rpm

Sampling time: 2, 4, 6, 8, and 10 minutes

Sampling Volume: 5 mL without replacement

Samples Analysis: HPLC/MS detection

Two Phase 1 clinical studies (1a/b) were carried out using the formulations described in Table 1. The details of the studies and in vivo results are detailed in U.S. patent Publication Nos. 20080147044, 20080268023, and 20090131479 expressly incorporated by reference herein.

TABLE 1
Formulations from Phase 1a/b Human Clinical Study

| | C1015001 | C1016001 | C1017001 |
|---|---|---|---|
| | (2.5 mcg) | (5 mcg) | (10 mcg) |
| | Composition, | Composition, | Composition, |
| Ingredient | % w/w | % w/w | % w/w |
| Sufentanil Citrate | 0.0680 | 0.136 | 0.273 |
| Mannitol | 73.90 | 73.86 | 73.7 |
| PEG 8000 | 15.0 | 15.0 | 15.0 |
| Polyox 303 | 3.0 | 3.0 | 3.0 |
| Pluronic F68 | 2.0 | 2.0 | 2.0 |
| Stearic Acid | 5.0 | 5.0 | 5.0 |
| Mg Stearate | 1.0 | 1.0 | 1.0 |
| Total | 100.0 | 100.0 | 100.0 |

An analysis of sufentanil release from solid tablets using this method yielded the results shown in Table 2 and FIG. 6.

TABLE 2
In Vitro Dissolution of Sufentanil Formulations

| | Mean % | Mean % | Mean % |
|---|---|---|---|
| | Dissolution | Dissolution | Dissolution |
| Time | C1015001 | C1016001 | C1017001 |
| (min.) | 2.5 mcg | 5 mcg | 10 mcg |
| 0 | 0 | 0 | 0 |
| 2 | 35 | 27 | 44 |
| 4 | 71 | 58 | 89 |
| 6 | 96 | 89 | 105 |
| 8 | 101 | 93 | 108 |
| 10 | 105 | 101 | 109 |

A number of formulations prepared with varying amounts of HPMC K4M were characterized by in vitro dissolution testing using a USP Type II dissolution apparatus, with a 100 mL vessel and mini paddles at 15 mm height.

The dissolution test was conducted under the following conditions:

Dissolution Media: 50 mM Acetate buffer at pH 4.5

Dissolution Volume: 50 mL

Temperature: 37° C.

Speed: 50 rpm

Sampling time: 4, 8, 12, 16 and 60 minutes

Sampling volume: 1 mL without replacement

Samples analysis: HPLC detection

TABLE 3
Description of HPMC Grades

| | Average Viscosity | % by weight |
|---|---|---|
| HPMC Grade | (mPas) | (range) |
| K4M | 4,000 | 3-12 |
| K15M | 15,000 | 0.5-3.2 |
| K100M | 100,000 | 0.1-0.5 |
| K100LV | 100 | approximately 10% |
| E5/E3 | 3-5 | approximately 10% |

TABLE 4
Dissolution of Sufentanil From Tablets with varied HPMC Content

| | Mean % | Mean % | Mean % | Mean % |
|---|---|---|---|---|
| | Dissolution | Dissolution | Dissolution | Dissolution |
| | ACRMT1502601 | Lot# 1476B001 | Lot# C1695001 | Lot# 080617 |
| | (15 mcg | (15 mcg | (15 mcg | (20 mcg |
| Time | sufentanil; | sufentanil; | sufentanil; | sufentanil; |
| (min.) | 1% HPMC) | 3% HPMC) | 6% HPMC) | 12% HPMC) |
| 4 | 98 | 95 | 42 | NA |
| 8 | 100 | 99 | 61 | NA |
| 12 | 99 | 100 | 76 | NA |
| 15 | NA | NA | NA | 67 |
| 30 | NA | NA | NA | 86 |
| 60 | 100 | 100 | 100 | 100 |

### Example 2

### Sufentanil Pharmacokinetic Study in Healthy Human Volunteer Subjects

An open-label, randomized, crossover, single-dose human clinical pharmacokinetic study was carried out in 12 healthy volunteer non-smoking male and female subjects aged 18 to 45 years of age with 5 treatment groups, as follows:
Treatment A: IV infusion Sufenta® (5 mcg) and single oral dose generic triazolam (125 mcg)
Treatment B: a single sublingual tablet containing 15 mcg sufentanil and 200 mcg triazolam administered by the sublingual route
Treatment C: a single tablet containing 15 mcg sufentanil administered by the sublingual route
Treatment D: a single tablet containing 15 mcg sufentanil administered by the buccal route
Treatment E: three 15 mcg sufentanil tablets administered orally (swallowed).

The objectives were: (1) to evaluate the pharmacokinetics of sublingual administration of a sufentanil and triazolam containing tablet, as compared to IV Sufenta IV and an oral generic commercially available from of triazolam; (2) to determine the pharmacokinetics of sufentanil following sublingual administration of a sufentanil and triazolam containing tablet relative to a tablet containing sufentanil alone in order to evaluate the effect of a formulation change; and (3) to determine the pharmacokinetics of administration of sufentanil by the (a) oral transmucosal/sublingual; (b) oral transmucosal/buccal; and (c) gastrointestinal (GI)/oral routes.

Study subjects received Treatment A, and then randomly received Treatments B, C, D and E. Serial blood samples were taken at 0 (pre-dose), 5, 10, 15, 20, 30, 45, 60, 90, 120, 180, 240, 360, 480, 600, and 720 minutes after the start of Treatments A, B and C for analysis of sufentanil (Treatments A, B, and C). Serial blood samples were taken at 0 (pre-dose), 20, 40, 60, 90, 120, 180, 240, 360, 480, 600, and 720 minutes after the start of Treatments D and E for analysis of sufentanil.

The erosion of tablets was monitored every 2 minutes in Treatments B, C, and D until completely eroded (no longer evident by visual examination).

Blood pressure, heart rate and respiratory rate was measured at various time intervals after the start of each treatment. Pulse oximetry was monitored continuously from pre-dose to 12 hours after the start of each treatment.

Non-compartmental PK parameters including the following, can be calculated based on the data presented in FIGS. 8A and B, which presents the mean sufentanil concentration in the plasma for each treatment group versus time:
(1) AUC₀₋ₜ [pg·h/mL]: Area under the plasma concentration time curve from time zero to time of the last quantifiable concentration following dosing calculated using the trapezoidal rule.
(2) AUC_{0-inf} [pg·h/mL]: Area under the plasma concentration time curve from time zero to time infinity.
(3) Cₘₐₓ [pg/mL]: maximum plasma concentration over the entire sampling period, directly obtained from the experimental data of plasma concentration versus time curves, without interpolation.
(4) Tₘₐₓ [h]: time to reach the maximum plasma concentration.
(5) t_{1/2}: Apparent terminal elimination half-life.

For the analysis of PK parameters derived from the sufentanil concentration data, an analysis of variance (ANOVA) model for a four-way crossover design was used to analyze the natural log-transformed AUC₀₋ₜ, AUC_{0-inf}, and Cₘₐₓ. This ANOVA model includes sequence, period, treatment, and period by treatment interaction fixed factors, and subject within sequence random factor. Estimates of the relative amount of sufentanil absorbed was calculated as the ratio of dose-normalized AUC_{0-inf}, value for Treatment B, D, E to Treatment C. Estimates of the absolute amount of sufentanil absorbed was calculated by multiplying each treatment's AUC_{0-inf} values and the clearance value derived from the reference IV treatment A (Sufenta).

### Example 3

### Phase 2 Clinical Study on Use of Sublingual Sufentanil in Patients Following Elective Unilateral Knee Replacement Surgery

A. Clinical Efficacy, Safety, and Tolerability

A multicenter, placebo-controlled, double-blind Phase 2 clinical study was carried out with a total of 101 patients (mean age=62.9, range 42-80), following elective unilateral knee replacement. Patients were randomized to receive placebo or an oral transmucosal sufentanil NanoTab containing 5 mcg, 10 mcg or 15 mcg of drug for treatment of post-operative pain after stabilization of pain levels in the post-operative care unit. Sufentanil was nurse administered sublingually as needed to treat pain at the patient's request, with a minimum re-dosing interval of 20 minutes. Patients were allowed to drop out of the study at any time. The primary efficacy endpoint was Sum of the Pain Intensity Difference SPID-12 (a cumulative measure of the difference in pain intensity over the 12-hour study compared to baseline).

Patients treated with 15 mcg Sufentanil NanoTabs experienced a significant reduction in pain intensity compared to placebo for the primary endpoint SPID-12 using the three alternative imputation methods, last observation carried forward “LOCF”, p=0.018, baseline observation carried forward “BOCF”, p=0.007, and worst observation carried forward “WOCF”, p=0.015 (Minkowitz et al., Reg. Anesth. Pain Med., 2010:8, American Society of Regional Anesthesia Spring Meeting, 2010). Only the 15 mcg dose met a key secondary endpoint, lower percentage of patient dropouts due to inadequate analgesia compared to placebo (p=0.006). No significant differences were observed among treatment groups for the overall incidence of adverse events with mild to moderate nausea and vomiting being the most common. There were no serious adverse events related to study drug.

B. Device Functionality.

A multicenter, open-label Phase 2 clinical study was carried out with a total of 30 patients (mean age 65.7; range 51-78) following elective unilateral knee replacement.

Patients self-administered 15 mcg doses of sufentanil NanoTabs sublingually as needed using a handheld multidose dispenser with a minimal re-dosing interval of 20 minutes which is referred to as a “Sufentanil NanoTab PCA System”. The primary efficacy endpoint was device functionality assessed as the proportion of patients who successfully completed the study without any type of system failure.

All enrolled patients (100%) completed the study without system failures or dosing errors of any kind, which included over 375 dispensed NanoTabs. Twenty-five patients (83.3%) reported the two highest scores on the 5-point Likert scale of overall patient's satisfaction with the System. All 30 enrolled patients indicated that they could handle the system easily and their user instructions were clear. All 30 enrolled patients indicated that the dosing tone was sufficiently loud and that the time required for dosing was just right. Twenty-seven patients (90%) indicated that the size and the shape of the dosing tip was just right. The majority of patients indicated that the other system features (weight, size, shape, and dose button function) were acceptable. The mean pain intensity difference (PID) scores increased from 0.3 at 15 minutes to 2.4 at 3 hours. After 3 hours, the mean PID were variable and ranged from 1.3 to 2.0. (See, Griffin et al., Reg. Anesth. Pain Med., 2010:10 American Society of Regional Anesthesia Spring Meeting, 2010)

This Phase 2 study confirmed the functionality, safety and efficacy of oral transmucosal sufentanil in management of acute moderate-to-severe post-operative pain following knee replacement surgery.

### Example 4

### Phase 2 Clinical Study on Use of Sublingual Sufentanil in Patients Following Abdominal Surgery

A multicenter, placebo-controlled, double-blind Phase 2 clinical study was carried out with a total of 88 patients (mean age=46.2, range 28-72) following major lower and upper abdominal surgery. Patients were randomized to receive placebo, 10 mcg or 15 mcg doses of Sufentanil NanoTabs for post-operative pain after stabilization of pain levels in the post-operative care unit. Study drug was nurse administered sublingually as needed to treat pain at the patient's request, with a minimum re-dosing interval of 20 minutes. Patients were allowed to drop out of the study at any time. The primary efficacy endpoint was Sum of the Pain Intensity Difference SPID-12 (a cumulative measure of the difference in pain intensity over the 12-hour study compared to baseline).

Patients receiving 10 mcg or 15 mcg of sufentanil NanoTabs experienced a significant reduction in pain intensity compared to placebo for the primary endpoint SPID-12 using the three alternative imputation methods “LOCF”, p<0.001 (10 and 15 mcg), “BOCF”, p=0.004 (10 mcg) and p<0.001 (15 mcg), and “WOCF”, p<0.001 (10 and 15 mcg). Furthermore, both the 10 mcg and 15 mcg dose met a key secondary endpoint, lower percentage of patient dropouts due to inadequate analgesia compared to placebo (p<0.001). There were no serious adverse events related to study drug.

This Phase 2 study demonstrates analgesic efficacy, safety and tolerability of oral transmucosal sufentanil in management of acute moderate-to-severe post-operative pain following major abdominal surgery.

### Example 5

### Phase 2 Clinical Trial for Treatment of Breakthrough Pain in Cancer Patients

A modification of a well-established protocol for treatment of cancer BTP described in Slatkin et al., www.SupportiveOncology.net, Vol. 5, Number 7, July/August 2007 was used in a multicenter, randomized, double-blind, placebo-controlled open-label titration to effective dose conducted with 36 patients. Patients meeting study enrollment criteria entered a 3-week open-label dose titration to establish a dose of oral transmucosal sufentanil, ranging from 20 to 80 mcg that provided adequate relief from cancer breakthrough pain without producing intolerable side effects. When the effective and tolerable dose was established, patients moved into the double-blind treatment phase.

The first dose of study medication was administered under the supervision of qualified study staff. Patients self-administered a 20 mcg dose of oral transmucosal sufentanil using a SDA and remained at the site for 2 hours. Respiratory rate, oxygen saturation, and sedation status was monitored after dosing by study staff.

Patients continued titration at home with a caregiver in attendance for at least 1 hour following each titration dose. Subsequent doses of the study medication were taken as needed for breakthrough pain, but the patient waited at least 2 hours after taking one dose of study medication before taking another study medication dose. Titration to the next higher dose of study medication (30, 40, 60 or 80 mcg) took place after at least 2 failed treatments at a current dose (i.e., adequate relief of breakthrough pain was not achieved), but patients waited at least 4 hours before titrating up to the next dose level. Patients who were able to establish a dose from 20 to 80 mcg that provided adequate analgesia with tolerable side effects returned to the clinic and were randomized to the double-blind treatment phase at that dose. The dose of oral transmucosal sufentanil established as optimal during the titration phase was the dose administered during the double-blind treatment phase.

Randomized patients received a kit of 10 blinded doses (7 active, 3 placebo). Patients were instructed to take a dose of their usual rescue medication if they did not experience adequate pain relief within 30 minutes after dosing with study medication. Patients were allowed to take a second dose of study medication at least 2 hours after a previous study medication dose, with a maximum of 4 doses per day of study medication during the double-blind phase.

An electronic diary was used to record pain intensity on an 11-point numerical rating scale (NRS), where 0=no pain, and 10=worst possible pain at baseline (prior to dose administration), and at 10, 15, 30, 45 and 60 minutes following each administration. The repeat cross-over design allowed patient to act as their own control

The primary endpoint was reported as the time-weighted summed pain intensity difference (SPID) over 30 minutes (SPID30). The pain intensity (PI) was evaluated at pre-dose, 10, 15, 30, 45, and 60 minutes post-dose.

The secondary endpoints included:
(1) The summed pain intensity difference at 10 minutes (SPID10), over 15 minutes (SPID15), over 45 minutes (SPID45), and over 60 minutes (SPID60);
(2) The pain intensity difference (PID) at 10, 15, 30, 45, and 60 minutes after each dose of study medication;
(3) Pain relief (PR) at 10, 15, 30, 45, and 60 minutes after each dose of study medication;
(4) Total pain relief (TOTPAR), defined as sum of the PR at 10 minutes, over 15 minutes (TOTPAR15), over 30 minutes (TOTPAR30), over 45 minutes (TOTPAR45), and over 60 minutes (TOTPAR60) after each dose of study medication;
(5) Global medication performance assessment (GMPA) at 60 minutes after each dose of study medication; and
(6) Use of rescue medication.

The statistical tests for the analysis of baseline variables and efficacy parameters was performed at the α=0.05 significance level using an ANOVA model. Patients from all study centers were be pooled for the analysis of efficacy data.

The primary endpoint indicated statistical significance at 30 minutes: time-weighted SPID-30, p<0.001; but not at 15 minutes, SPID-15, p=0.177.

The secondary endpoints indicated a pain intensity difference at each time point: PID-10: p=0.348; PID-15: p=0.027; time-weighted total pain relief at 10 minutes, p=0.049; at 15 minutes, p=0.009. There was no difference in any class of adverse events between active and placebo groups.

Although the foregoing invention has been described in some detail by way of illustration and example for purposes of clarity and understanding, it will be apparent to those skilled in the art that certain changes and modifications may be practiced. Various aspects of the invention have been achieved by a series of experiments, some of which are described by way of the following non-limiting examples. Therefore, the description and examples should not be construed as limiting the scope of the invention, which is delineated by the appended description of exemplary embodiments.

What is claimed is:

1. A pharmaceutical composition comprising sufentanil, having a bioavailability of from about 50% to about 90% following oral transmucosal administration and a bioavailability of from about 3% to about 8% following gastrointestinal (GI) administration.

2. The pharmaceutical composition according to claim 1, wherein the oral transmucosal bioavailability is greater than 60%.

3. The pharmaceutical composition according to claim 1, wherein the oral transmucosal bioavailability is greater than 55%.

4. The pharmaceutical composition according to claim 1, wherein the oral transmucosal bioavailability is greater than 50%.

5. The pharmaceutical composition according to claim 1, wherein said oral transmucosal administration is sublingual administration.

6. The pharmaceutical composition according to claim 1, wherein said oral transmucosal administration is buccal administration.

7. The pharmaceutical composition according to claim 1, wherein said GI administration is accomplished by swallowing.

8. The pharmaceutical composition according to claim 1, wherein said composition comprises from about 0.08% to about 2% sufentanil.

9. The pharmaceutical composition according to claim 1, wherein said composition comprises from about 5 mcg to about 200 mcg of sufentanil.

10. The pharmaceutical composition according to claim 1, wherein oral transmucosal administration results in a Tmax of from about 40 to about 50 minutes

11. The pharmaceutical composition according to claim 1, wherein oral transmucosal administration results in a Tmax with a coefficient of variation of less than 40%.

12. The pharmaceutical composition according to claim 1, wherein oral transmucosal administration results in a Cmax with a coefficient of variation of less than 40%.

13. The pharmaceutical composition according to claim 9, wherein said dose of sufentanil provides a mean AUC which is substantially dose proportional when administered to humans by the oral transmucosal route.

14. The pharmaceutical composition according to claim 9, wherein said dose of sufentanil provides a mean Cₘₐₓ which is substantially dose proportional when administered to humans by the oral transmucosal route.

15. The pharmaceutical composition according to claim 9, for use in the treatment of pain.

16. The pharmaceutical composition according to claim 15, wherein said pain is acute post-operative pain.

17. The pharmaceutical composition according to claim 15, wherein said pain is breakthrough pain.

18. The pharmaceutical composition according to claim 1, wherein said composition is a solid tablet comprising a bioadhesive material.

19. A multidose dispensing device comprising the composition according to claim 1.

20. A SDA comprising the composition according to claim 1.

21. A pharmaceutical formulation comprising sufentanil and from about 1% to 6% HPMC K4M, wherein when subjected to an in vitro dissolution test in a Type II USP dissolution apparatus, at least 70% of the total amount of sufentanil is released within 16 minutes.

22. The pharmaceutical formulation according to claim 21, comprising from about 1% to 3% HPMC K4M, wherein when subjected to an in vitro dissolution test in a Type II USP dissolution apparatus at least 70% of the total amount of sufentanil is released within 12 minutes.

23. The pharmaceutical formulation according to claim 21, comprising from about 1% to 3% HPMC K4M, wherein when subjected to an in vitro dissolution test in a Type II USP dissolution apparatus at least 70% of the total amount of sufentanil is released within 8 minutes.

24. The pharmaceutical formulation according to claim 21, having a bioavailability of from about 50% to about 90% following oral transmucosal administration and a bioavailability of from about 3% to about 8% following GI administration.

25. The pharmaceutical formulation according to claim 24, wherein the oral transmucosal bioavailability is greater than 60%.

26. The pharmaceutical formulation according to claim 24, wherein the oral transmucosal bioavailability is greater than 55%.

27. The pharmaceutical formulation according to claim 24, wherein the oral transmucosal bioavailability is greater than 50%.

28. The pharmaceutical formulation according to claim 24, wherein said oral transmucosal administration is sublingual administration.

29. The pharmaceutical formulation according to claim 24, wherein said oral transmucosal administration is buccal administration.

30. The pharmaceutical formulation according to claim 24, wherein said GI administration is accomplished by swallowing.

31. The pharmaceutical formulation according to claim 24, wherein said composition comprises from about 0.08% to about 2% sufentanil.

32. The pharmaceutical formulation according to claim 24, wherein said composition comprises from about 5 mcg to about 200 mcg of sufentanil.

33. A method for treating pain in a subject, comprising:
administering a sufentanil composition according to claim 1 to an oral mucosal surface of a subject wherein Tmax is from about 40 to about 50 minutes and the oral transmucosal bioavailability is greater than about 50% following said oral transmucosal administration.

34. A method for treating pain in a subject, comprising:
administering a sufentanil composition according to claim 21 to an oral mucosal surface of a subject wherein Tmax is from about 40 to about 50 minutes and the oral transmucosal bioavailability is greater than about 50% following said oral transmucosal administration.

35. The method according to claim 33, wherein said composition comprises from about 5 mcg to about 200 mcg of sufentanil.

36. The method according to claim 35, wherein pain is alleviated in said subject in from about 5 minutes to about 25 minutes following said administration.

37. The method according to claim 36, wherein pain is alleviated in said subject from about 10 minutes to about 20 minutes following said administration.
